(19) United States
(12) Patent Application Publication
(10) Pub. No.: US 2024/0269217 A1
(43) Pub. Date: Aug. 15, 2024

(54) FORSYTHIA SUSPENSA LEAF EXTRACT AND USE THEREOF FOR INCREASING ABUNDANCE OF AKK IN INTESTINAL TRACTS

(71) Applicant: ENLIGHTEN-AFRICA (SHENZHEN) BIOTECHNOLOGY CO., LTD., Shenzhen (CN)

(72) Inventors: Mingquan GUO, Shenzhen (CN); Guangwan HU, Shenzhen (CN)

(21) Appl. No.: 18/544,455

(22) Filed: Dec. 19, 2023

Related U.S. Application Data
(63) Continuation of application No. PCT/CN2021/140513, filed on Dec. 22, 2021.

(51) Int. Cl. A61K 36/634; A61P 1/00

(52) U.S. Cl. CPC A61K 36/634; A61P 1/00; A61K 2236/15; A61K 2236/331; A61K 2236/333; A61K 2236/39; A61K 2236/51; A61K 2236/55

(57) ABSTRACT

Disclosed are a preparation method for a Forsythia suspensa leaf extract and a use of the extract for increasing the abundance of AKK in intestinal tracts, which relate to the technical field of medicine. The preparation method for the Forsythia suspensa leaf extract includes: taking Forsythia suspensa leaves, adding a solvent at 90° C. or more for 10 to 40 minutes, filtering and then adding an extractant for extraction to obtain a first product; filtering the first product, taking a supernatant, adsorbing, eluting, and concentrating under reduced pressure to obtain an extract, and freeze-drying to obtain a second product; and dissolving the second product in water, carrying out chromatography, then eluting, collecting an eluate, concentrating under reduced pressure, and freeze-drying into a powder form to obtain the Forsythia suspensa leaf extract.

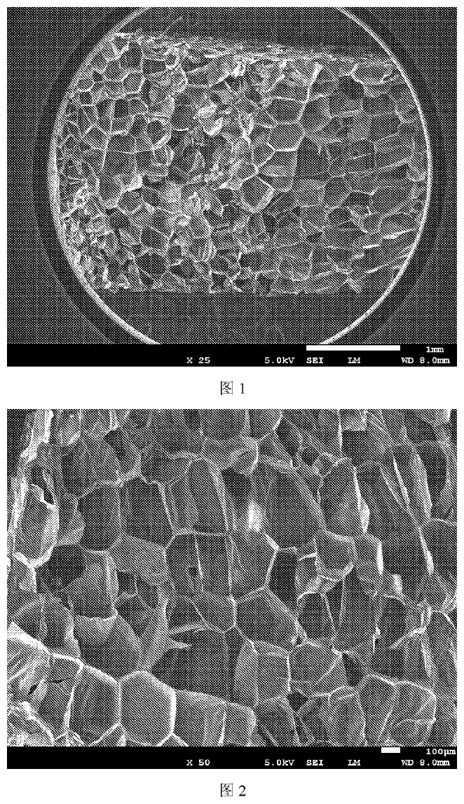

### CROSS-REFERENCE TO RELATED APPLICATIONS

The present application is a continuation of International Application No. PCT/CN2021/140513, with an international filing date of Dec. 22, 2021, which is based upon and claims priority to Chinese Patent Application No. 202110668873.X, filed on Jun. 16, 2021, the entire contents of all of which are incorporated herein by reference.

### TECHNICAL FIELD

The present invention relates to the technical field of medicine, in particular to a Forsythia suspensa leaf extract and its use for enhancing the abundance of AKK bacteria in intestinal tracts.

### BACKGROUND

With the advancement of genome sequencing technology, the significance of intestinal flora has become increasingly apparent. Intestinal flora plays a pivotal role in regulating host immunity, managing inflammatory responses, and overseeing energy metabolism. Akkermansia muciniphila (commonly referred to as AKK bacteria), identified and purified in 2004 by Dutch environmental microbiologist Antoon Akkermans and his colleagues, is an indigenous oval-shaped gram-negative anaerobe found in the human intestinal tracts. Constituting approximately 3-5% of the total major microbial communities in the human body, AKK bacteria's active content directly influences the thickness of the mucosal layer. A reduced abundance of AKK bacteria compromises the protective barrier function of the intestinal tract, making it more susceptible to the invasion of toxic substances and pathogens, thereby increasing the risk of disease. Studies have linked decreased AKK bacteria abundances to metabolic diseases such as inflammatory bowel disease and obesity. The emerging understanding of intestinal flora as a key regulator across metabolic, immune, and neuroendocrine pathways underscores the crucial role played by AKK bacteria in disease development. Its impact on host health has garnered increasing attention, positioning it as a potential target for controlling obesity, combating diabetes, and mitigating diseases related to cardiovascular and cerebrovascular system disorders.

Forsythia (Forsythia suspensa (Thunb.) Vahl) is a deciduous flowering shrub classified under the genus Forsythia in the Oleaceae family. Widely distributed in provinces such as Shandong, Henan, Hebei and Shanxi, this plant has drawn attention in modern research for its pharmaceutical components. Despite these findings, there are few studies discussing the effects of Forsythia extract on improving gastrointestinal function.

### SUMMARY

In response to the aforementioned issues, the present invention discloses a method for preparing Forsythia suspensa leaf extract.

Specifically, the embodiment of the present invention provides a preparation method for Forsythia suspensa leaf extract, which includes:
S1: Take Forsythia suspensa leaves, grind them, add a solvent at a temperature above 90° C. for 10 to 40 min, filter the mixture, then add an extractant and perform extraction for more than 6 hours to obtain a first product;
S2: Filter the first product, collect the supernatant, perform adsorption, elution, and concentrate the eluate under reduced pressure to obtain a second product;
S3: Dissolve the second product, conduct chromatography, elute, collect the eluate, and concentrate it under reduced pressure to obtain the Forsythia suspensa leaf extract.

Further, in step S1, the solvent is water and the extractant is an alcohol, preferably ethanol. Further, in step S2, the method of the adsorption is through a macroporous adsorption resin column.

Further, in step S2, the process of the eluting is as follows: first eluting with water, then eluting with 30%-60% ethanol, combining with an ethanol elution.

Further, in step S3, the volume ratio of the second product to the water is 1-10:5-50. Further, in step S3, the chromatography is preferably reversed phase silica column chromatography. Specifically, it's ODS-C-18 reversed phase silica column chromatography (WatersPrep150 LC, Waters BEH C18 VanGuard Pre-column, 5 UM, 19×250 mm, flow rate 5 mL/min).

The embodiment of the present invention also provides:
Forsythia suspensa leaf extract prepared by the above method.

The embodiment of the present invention also provides a use of Forsythia suspensa leaf extract obtained by the above preparation method for preparing a drug that promotes the abundance of the bacteria species Akkermansia muciniphila or the bacteria genus Akkermansia in the intestinal tract of animals.

Further, the drug also includes excipients.

Further, the abundance of AKK bacteria is the abundance in the intestinal tract. Further, the animals are mammal.

Furthermore, the Forsythia suspensa leaf extract (AFK-6801) provided by the embodiment of the present invention has shown a significant effect in promoting the proliferation of AKK bacteria in the intestinal tract according to animal experiments. The content of AKK bacteria can be substantially increased when the dosage is equal to or greater than 75 mg/kg.

### DETAILED DESCRIPTION OF THE EMBODIMENTS

To enhance clarity regarding the technical problem addressed, the technical solution proposed, and the resulting benefits, the present invention is elaborated upon in the following sections through specific embodiments. It is essential to note that the provided embodiments are intended for explanatory purposes and not to restrict the scope of the invention.

The embodiment of the present invention provides a preparation method for Forsythia suspensa leaf extract, including the following steps:
S1: Take Forsythia suspensa leaves, dry and grind them, add a solvent at a temperature above 90° C. for 10 to 40 min, filter, and then add an extractant for extraction for more than 6 hours to obtain a first product;
S2: Filter the first product, collect the supernatant, adsorb, elute, and concentrate the eluate under reduced pressure to obtain a second product.
S3: Dissolve the second product, conduct chromatography, elute, collect an eluate, and concentrate it under reduced pressure to obtain the Forsythia suspensa leaf extract.

Preferably, in step S1, the drying and grinding includes either air drying or freeze-drying, preferably natural air drying.

Preferably, in step S1, the solvent is water, and the extractant is an alcohol (e.g., methanol, ethanol, propanol), preferably ethanol, and further preferably 75% edible ethanol.

Specifically, the amount of water in the solvent is 5-20 times the volume of the solid obtained after grinding, with a filtration step included after water treatment.

Extraction with the extractant takes more than 5 hours, at normal temperature or 4° C.

Preferably, in step S2, the method of the adsorption is through a macroporous adsorption resin column, preferably, HP-20 macroporous adsorption resin column. Preferably, in step S2, the process of the eluting is: first eluting with water, then eluting with 30%-60% ethanol, combining with an ethanol elution.

Further preferably, this step also includes the operation of concentrating under reduced pressure.

Preferably, in step S3, the volume ratio of the second product to water is 1-10:5-50.

Preferably, in step S3, the chromatography is preferably reversed phase silica column chromatography. Specifically, it's ODS-C-18 reversed phase silica column chromatography (WatersPrep150 LC, Waters BEH C18 VanGuard Pre-column, 5 UM, 19×250 mm, flow rate 5 mL/min).

The embodiment of the present invention further provides:
A use of Forsythia suspensa leaf extract obtained by the above preparation method.
A use of Forsythia suspensa leaf extract for preparing a drug to increase the abundance of AKK bacteria in vivo in animals.

Further, the drug is an oral drug that could be a paste, liquid, syrup, tablet, capsule, or other suitable dosage form, and further preferably, the drug also includes excipients.

Further, the abundance of AKK bacteria is the abundance in the intestinal tract. Further preferably, the animals are mammal.
The Forsythia suspensa leaf extract (AFK-6801) has demonstrated a significant effect in promoting the proliferation of AKK bacteria in the intestinal tract based on animal experiments.

For further details, specific embodiments are provided below.

### Example 1: Preparation Method of Forsythia suspensa Leaf Extract

The preparation method of Forsythia suspensa leaf extract included the following steps:
(1) taking Forsythia suspensa leaves, drying them in natural air, grinding them, adding water 10 times their volume to boil them for 30 min, filtering them with gauze, adding 95% edible ethanol, adjusting the ethanol concentration to 75%, and placing them in a refrigerator at 4° C. overnight to obtain a first product;
(2) filtering the first product, taking a supernatant, and adding it to a HP-20 macroporous adsorption resin column for chromatography with a sample amount that is 30% of the column volume, letting it stand for 4 h, first eluting it with water for 3 column volumes, then eluting it with 40% ethanol for 3 column volumes, combining with 40% ethanol eluate, concentrating it under reduced pressure on a rotary evaporator to obtain an extract, the pressure for concentrating under reduced pressure was 10 kPa and the temperature was 65° C. Then freeze-drying to obtain a second product (crude extract).
(3) dissolving the second product with water at a volume ratio of 1:5, adopting ODS-C-18 reversed phase silica column chromatography (WatersPrep150 LC, Waters BEH C18 VanGuard Pre-column, 5 μM, 19×250 mm, flow rate 5 mL/min) with a sample size of 1 mL column. Eluting with 20% and 40% methanol water successively, collecting an eluate of 40% methanol water, concentrating it under reduced pressure, and freeze-drying it into powder (yield 0.2%, purity 98.8%) to obtain final extract.
(4) the obtained product was named as AFK-6801, and its nuclear magnetic data were as follows: brown solid powder, soluble in methanol, fluorescent at 254 nm, yellow spots on I₂, yellow to black on heating with 5% H₂SO₄-EtOH. The molecular formula was C₂₅H₃₈O₁₆, ESI-MS: m/z 594.2 [M-H]⁻.

Nuclear magnetic resonance spectroscopy of the extract showed that the AFK-6801 compound fluoresced at 254 nm, showed yellow spots on I₂, showed yellow to black on heating with 5% H₂SO₄-EtOH, ESI-MS: m/z 594.2 [M-H]⁻. The attribution data of C and H are shown in Table 1:

TABLE 1
The attribution data of hydrogen and carbon spectra
of AFK-6801 compounds by nuclear magnetic resonance

| Position | δ_{H} (mult, J in Hz) | δ_{C} |
|---|---|---|
| 1 | | 131.7 |
| 2 | 6.69 (d, J = 2.0) | 116.6 |
| 3 | | 146.1 |
| 4 | | 144.7 |
| 5 | 6.67(d, J = 8.0) | 117.6 |
| 6 | 6.55 (dd, J = 8.0, 2.0) | 121.5 |
| β | 2.78(m) | 36.6 |
| α | 4.04(m) | 72.4 |
| Glc-1′ | 4.46(d, J = 8.0) | 103.2 |
| Glc-2′ | 3.40 (dd, J = 8.0, 9.0) | 79.9 |
| Glc-3′ | 3.55 (m) | 71.6 |
| Glc-4′ | 3.42(m) | 78.0 |
| Glc-5′ | 3.22(m) | 78.0 |
| Glc-6′ | 3.88(dd, J = 12.0, 2.2) | 68.9 |
| | 3.70 (dd, J = 12.0, 4.8) | |
| Rha-1″ | 5.02 (d, J = 8.0) | 102.8 |
| Rha-2″ | 3.68(m) | 72.4 |
| Rha-3″ | 3.32(m) | 72.4 |
| Rha-4″ | 3.55(m) | 73.7 |
| Rha-5″ | 3.70(m) | 70.5 |
| Rha-6″ | 1.12(s) | 18.6 |
| Fur-1″′ | 5.52(d, J = 7.6) | 103.1 |
| Fur-2″′ | 4.03(m) | 77.5 |
| Fur-3″′ | | 81.6 |
| Fur-4″′ | 3.71(d, J = 12.0, 2.0) | 74.2 |
| | 3.92(d, J = 12.0, 2.0) | |
| Fur-5″′ | 3.72 (d, J = 12.0, 2.4) | 64.5 |
| | 2.73(d, J = 12.0, 2.4) | |

The structural formula of the identified compound AFK-6801 is shown in formula 1, and is named as Latifoside I (3,4-dihydroxyphenylethanol-β-O-α-1-rhamnosyl-(1→2)-[β-D-xyfuranosyl-(1→6)]-β-d-glucoside), and the structural formula is as follows:

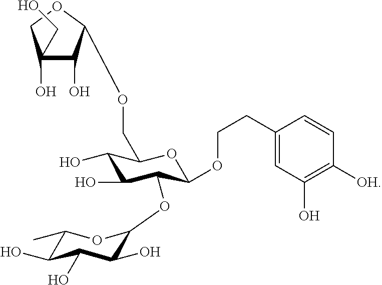

### Example 2: Preparation Method of Forsythia suspensa Leaf Extract

In this embodiment, the deviation from Embodiment 1 involves substituting 75% ethanol with methanol in step (1). The subsequent operational steps remained consistent, and the results indicated a close resemblance in the extract composition to that of Embodiment 1.

### Example 3: Evaluating the Promoting Effect of Forsythia suspensa Leaf Extract on the Proliferation of AKK Bacteria in the Intestinal Tract of Animals

### 1. Experimental Methods

### Mice and Administration

A total of 36 wild-type male C57BL/6J (WT) mice, aged 6 weeks, were procured from Zhejiang Vital River Laboratory Animal Technology Co. Ltd. The mice were housed in a temperature-controlled animal room (20-22° C.) with a 12-hour light/dark cycle and underwent adaptive feeding for 7 days.

The Forsythia suspensa leaf extract prepared in Embodiment 1 was dissolved in water, resulting in a 10 mg/ml solution.

The C57BL/6 mice were fed SPF-grade breeding feed and provided free access to water throughout the experiment. After 7 days of adaptive feeding, the C57BL/6 mice were randomly divided into four groups (n=9 in each group): (1) the normal control group (CW), (2) the high-dose administration group (D-H 100 mg/kg), (3) the medium-dose administration group (D-L, 75 mg/kg), and (4) the low-dose administration group (D-L, 50 mg/kg). The normal control group (1) received purified water by gavage based on body weight, while the administration groups (2)-(4) received gavage according to their respective dosage for 8 weeks.

Analysis of Intestinal Bacteria 16S rRNA

On the final day of the experimental administration, feces from each mouse were collected and stored in a 2 mL centrifuge tube for immediate examination.

The total DNA of the fecal samples was extracted, and primers were designed based on the conserved region. Sequencing adapters were added to the primers for PCR amplification and purification. The resulting products were then purified, quantified, and homogenized to create a sequencing library. The constructed library underwent initial quality inspection, and only the qualified libraries were sequenced using Illumina HiSeq 2500.

The raw image data files obtained from high-throughput sequencing platforms, such as Illumina HiSeq, were converted into original sequencing sequences (Sequenced Reads) through Base Calling analysis. The Qiime1.91 standardized process and Open_Reference algorithm were employed to perform OTU clustering at 97% similarity, significantly enhancing the efficiency of strain annotation. Finally, Biom data, containing sample names, strain annotations, and abundance information, was generated through comparison with the GreenGene database.

### Statistical Analysis

The experimental data were expressed as mean±SD. One-way ANOVA analysis was conducted using Graphpad Prism 7.0 software to explore differences among the data. Statistical significance was denoted as *p<0.05, indicating a significant difference, and **p<0.01, indicating an extremely significant difference.

### Experimental Results of Intestinal Flora Analysis

The results of the intestinal flora analysis are presented in Table 2 below.

TABLE 2
Effects of Forsythia suspensa leaf extract on the phylum level of intestinal flora

| | | D-H | D-M | D-L | CW |
|---|---|---|---|---|---|
| Legend | Taxonomy | % | % | % | % |
| | Unassigned; Other | 0.0% | 0.2% | 0.0% | 0.0% |
| | k_{——}Bacteria; p_{——}Actinobacteria | 0.8% | 2.1% | 1.9% | 2.2% |
| | k_{——}Bacteria; p_{——}Bacteroidetes | 15.6% | 5.4% | 9.7% | 27.3% |
| | k_{——}Bacteria; p_{——}Chlamydiae | 0.0% | 0.3% | 0.4% | 0.1% |
| | k_{——}Bacteria; p_{——}Deferribacteres | 0.1% | 1.4% | 0.5% | 0.1% |
| | k_{——}Bacteria; p_{——}Firmicutes | 35.7% | 77.9% | 66.4% | 61.4% |
| | k_{——}Bacteria; p_{——}Proteobacteria | 18.7% | 10.8% | 20.2% | 7.7% |
| | k_{——}Bacteria; p_{——}TM7 | 0.0% | 0.9% | 0.7% | 0.8% |
| | k_{——}Bacteria; p_{——}Tenericutes | 0.0% | 0.0% | 0.2% | 0.3% |
| | k_{——}Bacteria; p_{——}Verrucomicrobia | 29.1% | 0.9% | 0.0% | 0.0% |

As evident from the table above, both the high-dose administration group (D-H, 100 mg/kg) and the medium-dose administration group (D-L, 75 mg/kg) exhibit significant enhancements in the number of microflora within the phylum Verrucomicrobia. Notably, the high-dose group demonstrated a remarkable increase rate of 29.1% compared to the control group.

Furthermore, microflora below the phylum Verrucomicrobia were selected for detection and analysis, with the results presented in Table 3.

TABLE 3
Effects of Forsythia suspensa leaf extract on the class level of intestinal flora

| | | D-H | D-M | D-L | CW |
|---|---|---|---|---|---|
| Legend | Taxonomy | % | % | % | % |
| | Unassigned; Other; Other | 0.0% | 0.2% | 0.0% | 0.0% |
| | k_{——}Bacteria; p_{——}Actinobacteria; c_{——}Actinobacteria | 0.0% | 0.0% | 0.0% | 0.2% |
| | k_{——}Bacteria; p_{——}Actinobacteria; c_{——}Coriobacteriia | 0.8% | 2.1% | 1.9% | 2.0% |
| | k_{——}Bacteria; p_{——}Bacteroidetes; c_{——}Bacteroidia | 15.6% | 5.4% | 9.7% | 27.3% |
| | k_{——}Bacteria; p_{——}Chlamydiae; c_{——}Chlamydiia | 0.0% | 0.3% | 0.4% | 0.1% |
| | k_{——}Bacteria; p_{——}Deferribacteres; c_{——}Deferribacteres | 0.1% | 1.4% | 0.5% | 0.1% |
| | k_{——}Bacteria; p_{——}Firmicutes; c_{——}Bacilli | 0.4% | 10.6% | 15.6% | 6.4% |
| | k_{——}Bacteria; p_{——}Firmicutes; c_{——}Clostridia | 20.3% | 57.6% | 44.0% | 40.4% |
| | k_{——}Bacteria; p_{——}Firmicutes; c_{——}Erysipelotrichi | 15.0% | 9.7% | 6.7% | 14.6% |
| | k_{——}Bacteria; p_{——}Proteobacteria; c_{——}Betaproteobacteria | 1.9% | 0.0% | 0.0% | 1.1% |
| | k_{——}Bacteria; p_{——}Proteobacteria; c_{——}Deltaproteobacteria | 0.7% | 7.8% | 19.4% | 2.1% |
| | k_{——}Bacteria; p_{——}Proteobacteria; c_{——}Epsilonproteobacteria | 0.0% | 2.6% | 0.7% | 0.8% |
| | k_{——}Bacteria; p_{——}Proteobacteria; c_{——}Gammaproteobacteria | 16.0% | 0.3% | 0.1% | 3.6% |
| | k_{——}Bacteria; p_{——}TM7; c_{——}TM7-3 | 0.0% | 0.9% | 0.7% | 0.8% |
| | k_{——}Bacteria; p_{——}Tenericutes; c_{——}Mollicutes | 0.0% | 0.0% | 0.2% | 0.3% |
| | k_{——}Bacteria; p_{——}Verrucomicrobia; c_{——}Verrucomicrobiae | 29.1% | 0.9% | 0.0% | 0.0% |

As observed in the table above, both the high-dose administration group (D-H, 100 mg/kg) and the medium-dose administration group (D-L, 75 mg/kg) demonstrate significant enhancements in the number of microflora within the class Verrucomicrobieae under the phylum Verrucomicrobia. Notably, the high-dose group exhibited a remarkable increase rate of 29.1% compared to the control group.

Subsequent experiments focused on the microflora at the order level under Verrucomicrobiae, with the results presented in Table 4.

TABLE 4
Effects of Forsythia suspensa leaf extract on the order level of intestinal flora

| | | D-H | D-M | D-L | CW |
|---|---|---|---|---|---|
| Legend | Taxonomy | % | % | % | % |
| | Unassigned; Other; Other; Other | 0.0% | 0.2% | 0.0% | 0.0% |
| | k_{——}Bacteria; p_{——}Actinobacteria; c_{——}Coriobacteriia; o_{——}Coriobacteriales | 0.8% | 2.1% | 1.9% | 2.0% |
| | k_{——}Bacteria; p_{——}Bacteroidetes; c_{——}Bacteroidia; o_{——}Bacteroidales | 15.6% | 5.4% | 9.7% | 27.3% |
| | k_{——}Bacteria; p_{——}Chlamydiae; c_{——}Chlamydiia; o_{——}Chlamydiales | 0.0% | 0.3% | 0.4% | 0.1% |
| | k_{——}Bacteria; p_{——}Deferribacteres; c_{——}Deferribacteres; o_{——}Deferribacterales | 0.1% | 1.4% | 0.5% | 0.1% |
| | k_{——}Bacteria; p_{——}Firmicutes; c_{——}Bacilli; o_{——}Lactobacillales | 0.4% | 10.6% | 15.6% | 6.4% |
| | k_{——}Bacteria; p_{——}Firmicutes; c_{——}Bacilli; o_{——}Turicibacterales | 0.0% | 0.0% | 0.0% | 0.0% |
| | k_{——}Bacteria; p_{——}Firmicutes; c_{——}Clostridia; o_{——}Clostridiales | 20.3% | 57.6% | 44.0% | 40.4% |
| | k_{——}Bacteria; p_{——}Firmicutes; c_{——}Erysipelotrichi; o_{——}Erysipelotrichales | 15.0% | 9.7% | 6.7% | 14.6% |
| | k_{——}Bacteria; p_{——}Proteobacteria; c_{——}Betaproteobacteria; o_{——}Burkholderiales | 1.9% | 0.0% | 0.0% | 1.1% |
| | k_{——}Bacteria; p_{——}Proteobacteria; c_{——}Deltaproteobacteria; o_{——}Desulfovibrionales | 0.7% | 7.8% | 19.4% | 2.1% |
| | k_{——}Bacteria; p_{——}Proteobacteria; c_{——}Epsilonproteobacteria; o_{——}Campylobacterales | 0.0% | 2.6% | 0.7% | 0.8% |
| | k_{——}Bacteria; p_{——}Proteobacteria; c_{——}Gammaproteobacteria; o_{——}Enterobacteriales | 16.0% | 0.1% | 0.1% | 3.6% |
| | k_{——}Bacteria; p_{——}TM7; c_{——}TM7-3; o_{——}CW040 | 0.0% | 0.9% | 0.7% | 0.8% |
| | k_{——}Bacteria; p_{——}Tenericutes; c_{——}Mollicutes; o_{——}Anaeroplasmatales | 0.0% | 0.0% | 0.0% | 0.2% |
| | k_{——}Bacteria; p_{——}Tenericutes; c_{——}Mollicutes; o_{——}RF39 | 0.0% | 0.0% | 0.2% | 0.2% |
| | k_{——}Bacteria; p_{——}Verrucomicrobia; c_{——}Verrucomicrobiae; o_{——}Verrucomicrobiales | 29.1% | 0.9% | 0.0% | 0.0% |

As evident from the table above, both the high-dose administration group (D-H, 100 mg/kg) and the medium-dose administration group (D-L, 75 mg/kg) exhibit significant enhancements in the number of microflora within the order Verrucomicrobiales under the phylum Verrucomicrobieae. Notably, the high-dose group demonstrated a remarkable increase rate of 29.1% compared to the control group.

Further analysis was conducted on the strains included in the microflora of this order, and the results are presented in Table 5.

TABLE 5
Effects of Forsythia suspensa leaf extract on the family level of intestinal flora

| | | D-H | D-L | D-M | CW |
|---|---|---|---|---|---|
| Legend | Taxonomy | % | % | % | % |
| | Unassigned; Other; Other; Other; Other | 0.0% | 0.2% | 0.0% | 0.0% |
| | k_{——}Bacteria; p_{——}Actinobacteria; c_{——}Coriobacteriia; o_{——}Coriobacteriales; | 0.8% | 2.1% | 1.9% | 2.0% |
| | f_{——}Coriobacteriaceae | | | | |
| | k_{——}Bacteria; p_{——}Bacteroidetes; c_{——}Bacteroidia; o_{——}Bacteroidales; f_{——} | 0.1% | 0.2% | 1.3% | 0.4% |
| | k_{——}Bacteria; p_{——}Bacteroidetes; c_{——}Bacteroidia; o_{——}Bacteroidales; | 13.3% | 1.0% | 0.8% | 0.3% |
| | f_{——}Bacteroidaceae | | | | |
| | k_{——}Bacteria; p_{——}Bacteroidetes; c_{——}Bacteroidia; o_{——}Bacteroidales; | 2.1% | 0.1% | 0.0% | 0.1% |
| | f_{——}Porphyromonadaceae | | | | |
| | k_{——}Bacteria; p_{——}Bacteroidetes; c_{——}Bacteroidia; o_{——}Bacteroidales; | 0.0% | 0.5% | 1.1% | 0.8% |
| | f_{——}Rikenellaceae | | | | |
| | k_{——}Bacteria; p_{——}Bacteroidetes; c_{——}Bacteroidia; o_{——}Bacteroidales; | 0.1% | 3.5% | 5.2% | 21.7% |
| | f_{——}S24-7 | | | | |
| | k_{——}Bacteria; p_{——}Bacteroidetes; c_{——}Bacteroidia; o_{——}Bacteroidales; | 0.0% | 0.1% | 1.0% | 0.4% |
| | f_{——}[Odoribacteraceae] | | | | |
| | k_{——}Bacteria; p_{——}Bacteroidetes; c_{——}Bacteroidia; o_{——}Bacteroidales; | 0.0% | 0.0% | 0.2% | 3.4% |
| | f_{——}[Paraprevotellaceae] | | | | |
| | k_{——}Bacteria; p_{——}Chlamydiae; c_{——}Chlamydiia; o_{——}Chlamydiales; | 0.0% | 0.3% | 0.4% | 0.1% |
| | f_{——}Chlamydiaceae | | | | |
| | k_{——}Bacteria; p_{——}Deferribacteres; c_{——}Deferribacteres; | 0.1% | 1.4% | 0.5% | 0.1% |
| | o_{——}Deferribacterales; f_{——}Deferribacteraceae | | | | |
| | k_{——}Bacteria; p_{——}Firmicutes; c_{——}Bacilli; o_{——}Lactobacillales; | 0.1% | 0.0% | 0.2% | 0.0% |
| | f_{——}Enterococcaceae | | | | |
| | k_{——}Bacteria; p_{——}Firmicutes; c_{——}Bacilli; o_{——}Lactobacillales; | 0.1% | 2.4% | 11.8% | 6.3% |
| | f_{——}Lactobacillaceae | | | | |
| | k_{——}Bacteria; p_{——}Firmicutes; c_{——}Bacilli; o_{——}Lactobacillales; | 0.2% | 8.2% | 3.6% | 0.1% |
| | f_{——}Streptococcaceae | | | | |
| | k_{——}Bacteria; p_{——}Firmicutes; c_{——}Clostridia; o_{——}Clostridiales; Other | 0.0% | 0.1% | 0.1% | 0.1% |
| | k_{——}Bacteria; p_{——}Firmicutes; c_{——}Clostridia; o_{——}Clostridiales; f_{——} | 0.1% | 19.6% | 20.4% | 22.9% |
| | k_{——}Bacteria; p_{——}Firmicutes; c_{——}Clostridia; o_{——}Clostridiales; | 0.0% | 0.3% | 0.1% | 0.4% |
| | f_{——}Clostridiaceae | | | | |
| | k_{——}Bacteria; p_{——}Firmicutes; c_{——}Clostridia; o_{——}Clostridiales; | 7.0% | 17.7% | 10.7% | 9.5% |
| | f_{——}Lachnospiraceae | | | | |
| | k_{——}Bacteria; p_{——}Firmicutes; c_{——}Clostridia; o_{——}Clostridiales; | 13.2% | 18.8% | 11.2% | 6.9% |
| | f_{——}Ruminococcaceae | | | | |
| | k_{——}Bacteria; p_{——}Firmicutes; c_{——}Clostridia; o_{——}Clostridiales; | 0.0% | 0.5% | 0.7% | 0.4% |
| | f_{——}Veillonellaceae | | | | |
| | k_{——}Bacteria; p_{——}Firmicutes; c_{——}Clostridia; o_{——}Clostridiales; | 0.0% | 0.5% | 0.3% | 0.0% |
| | f_{——}[Mogibacteriaceae] | | | | |
| | k_{——}Bacteria; p_{——}Firmicutes; c_{——}Erysipelotrichi; o_{——}Erysipelotrichales; | 15.0% | 9.7% | 6.7% | 14.6% |
| | f_{——}Erysipelotrichaceae | | | | |
| | k_{——}Bacteria; p_{——}Proteobacteria; c_{——}Alphaproteobacteria; | 0.0% | 0.1% | 0.0% | 0.0% |
| | o_{——}Rickettsiales; f_{——} | | | | |
| | k_{——}Bacteria; p_{——}Proteobacteria; c_{——}Betaproteobacteria; | 1.9% | 0.0% | 0.0% | 1.1% |
| | o_{——}Burkholderiales; f_{——}Alcaligenaceae | | | | |
| | k_{——}Bacteria; p_{——}Proteobacteria; c_{——}Deltaproteobacteria; | 0.7% | 7.8% | 19.4% | 2.1% |
| | o_{——}Desulfovibrionales; f_{——}Desulfovibrionaceae | | | | |
| | k_{——}Bacteria; p_{——}Proteobacteria; c_{——}Epsilonproteobacteria; | 0.0% | 2.6% | 0.7% | 0.8% |
| | o_{——}Campylobacterales; f_{——}Helicobacteraceae | | | | |
| | k_{——}Bacteria; p_{——}Proteobacteria; c_{——}Gammaproteobacteria; | 16.0% | 0.1% | 0.1% | 3.6% |
| | o_{——}Enterobacteriales; f_{——}Enterobacteriaceae | | | | |
| | k_{——}Bacteria; p_{——}TM7; c_{——}TM7-3; o_{——}CW040; f_{——}F16 | 0.0% | 0.9% | 0.7% | 0.8% |
| | k_{——}Bacteria; p_{——}Verrucomicrobia; c_{——}Verrucomicrobiae; | 29.1% | 0.9% | 0.0% | 0.0% |
| | o_{——}Verrucomicrobiales; f_{——}Verrucomicrobiaceae | | | | |

As evident from the table above, both the high-dose administration group (D-H, 100 mg/kg) and the medium-dose administration group (D-L, 75 mg/kg) exhibit significant enhancements in the number of microflora within the family Verrucomicrobiaceae. Notably, the high-dose group demonstrated a remarkable increase rate of 29.1% compared to the control group.

Subsequent detection and analysis were conducted for microflora within each genus in this family.

TABLE 6
Effects of Forsythia suspensa leaf extract on the genus level of intestinal flora

| | | D-H | D-L | D-M | CW |
|---|---|---|---|---|---|
| Legend | Taxonomy | % | % | % | % |
| | k_{——}Bacteria; p_{——}Actinobacteria; c_{——}Coriobacteriia; | 0.0% | 0.4% | 0.6% | 0.7% |
| | o_{——}Coriobacteriales; f_{——}Coriobacteriaceae; g_{——} | | | | |
| | k_{——}Bacteria; p_{——}Actinobacteria; c_{——}Coriobacteriia; | 0.1% | 1.6% | 1.2% | 0.7% |
| | o_{——}Coriobacteriales; f_{——}Coriobacteriaceae; g_{——}Adlercreutzia | | | | |
| | k_{——}Bacteria; p_{——}Bacteroidetes; c_{——}Bacteroidia; | 0.1% | 0.2% | 1.3% | 0.4% |
| | o_{——}Bacteroidales; f_{——}; g_{——} | | | | |
| | k_{——}Bacteria; p_{——}Bacteroidetes; c_{——}Bacteroidia; | 13.3% | 1.0% | 0.8% | 0.3% |
| | o_{——}Bacteroidales; f_{——}Bacteroidaceae; g_{——}Bacteroides | | | | |
| | k_{——}Bacteria; p_{——}Bacteroidetes; c_{——}Bacteroidia; | 2.1% | 0.1% | 0.0% | 0.1% |
| | o_{——}Bacteroidales; f_{——}Porphyromonadaceae; g_{——}Parabacteroides | | | | |
| | k_{——}Bacteria; p_{——}Bacteroidetes; c_{——}Bacteroidia; | 0.0% | 0.5% | 0.6% | 0.2% |
| | o_{——}Bacteroidales; f_{——}Rikenellaceae; g_{——} | | | | |
| | k_{——}Bacteria; p_{——}Bacteroidetes; c_{——}Bacteroidia; | 0.1% | 3.5% | 5.2% | 21.7% |
| | o_{——}Bacteroidales; f_{——}S24-7; g_{——} | | | | |
| | k_{——}Bacteria; p_{——}Bacteroidetes; c_{——}Bacteroidia; | 0.0% | 0.0% | 1.0% | 0.4% |
| | o_{——}Bacteroidales; f_{——}[Odoribacteraceae]; g_{——}Odoribacter | | | | |
| | k_{——}Bacteria; p_{——}Bacteroidetes; c_{——}Bacteroidia; | 0.0% | 0.0% | 0.2% | 3.4% |
| | o_{——}Bacteroidales; f_{——}[Paraprevotellaceae]; g_{——}[Prevotella] | | | | |
| | k_{——}Bacteria; p_{——}Chlamydiae; c_{——}Chlamydiia; o_{——}Chlamydiales; | 0.0% | 0.3% | 0.4% | 0.1% |
| | f_{——}Chlamydiaceae; g_{——}Chlamydia | | | | |
| | k_{——}Bacteria; p_{——}Deferribacteres; c_{——}Deferribacteres; | 0.1% | 1.4% | 0.5% | 0.1% |
| | o_{——}Deferribacterales; f_{——}Deferribacteraceae; g_{——}Mucispirillum | | | | |
| | k_{——}Bacteria; p_{——}Firmicutes; c_{——}Bacilli; o_{——}Lactobacillales; | 0.1% | 2.4% | 11.8% | 6.3% |
| | o_{——}Lactobacillaceae; g_{——}Lactobacillus | | | | |
| | k_{——}Bacteria; p_{——}Firmicutes; c_{——}Bacilli; o_{——}Lactobacillales; | 0.1% | 7.8% | 3.4% | 0.0% |
| | f_{——}Streptococcaceae; g_{——}Lactococcus | | | | |
| | k_{——}Bacteria; p_{——}Firmicutes; c_{——}Bacilli; o_{——}Lactobacillales; | 0.0% | 0.4% | 0.2% | 0.1% |
| | f_{——}Streptococcaceae; g_{——}Streptococcus | | | | |
| | k_{——}Bacteria; p_{——}Firmicutes; c_{——}Clostridia; o_{——}Clostridiales; f_{——}; g_{——} | 0.1% | 19.6% | 20.4% | 22.9% |
| | k_{——}Bacteria; p_{——}Firmicutes; c_{——}Clostridia; o_{——}Clostridiales; | 0.1% | 0.5% | 0.1% | 0.1% |
| | f_{——}Lachnospiraceae; Other | | | | |
| | k_{——}Bacteria; p_{——}Firmicutes; c_{——}Clostridia; o_{——}Clostridiales; | 2.6% | 3.1% | 1.9% | 5.0% |
| | f_{——}Lachnospiraceae; g_{——} | | | | |
| | k_{——}Bacteria; p_{——}Firmicutes; c_{——}Clostridia; o_{——}Clostridiales; | 3.3% | 0.1% | 0.0% | 0.0% |
| | f_{——}Lachnospiraceae; g_{——}Blautia | | | | |
| | k_{——}Bacteria; p_{——}Firmicutes; c_{——}Clostridia; o_{——}Clostridiales; | 0.0% | 1.0% | 0.2% | 0.2% |
| | f_{——}Lachnospiraceae; g_{——}Coprococcus | | | | |
| | k_{——}Bacteria; p_{——}Firmicutes; c_{——}Clostridia; o_{——}Clostridiales; | 0.2% | 6.1% | 5.9% | 1.2% |
| | f_{——}Lachnospiraceae; g_{——}Dorea | | | | |
| | k_{——}Bacteria; p_{——}Firmicutes; c_{——}Clostridia; o_{——}Clostridiales; | 0.0% | 2.0% | 0.4% | 0.2% |
| | f_{——}Lachnospiraceae; g_{——}Roseburia | | | | |
| | k_{——}Bacteria; p_{——}Firmicutes; c_{——}Clostridia; o_{——}Clostridiales; | 0.9% | 4.9% | 2.3% | 2.7% |
| | f_{——}Lachnospiraceae; g_{——}[Ruminococcus] | | | | |
| | k_{——}Bacteria; p_{——}Firmicutes; c_{——}Clostridia; o_{——}Clostridiales; | 11.9% | 8.3% | 1.9% | 2.3% |
| | f_{——}Ruminococcaceae; g_{——} | | | | |
| | k_{——}Bacteria; p_{——}Firmicutes; c_{——}Clostridia; o_{——}Clostridiales; | 0.7% | 9.1% | 8.5% | 2.8% |
| | f_{——}Ruminococcaceae; g_{——}Oscillospira | | | | |
| | k_{——}Bacteria; p_{——}Firmicutes; c_{——}Clostridia; o_{——}Clostridiales; | 0.1% | 1.2% | 0.5% | 1.6% |
| | f_{——}Ruminococcaceae; g_{——}Ruminococcus | | | | |
| | k_{——}Bacteria; p_{——}Firmicutes; c_{——}Clostridia; o_{——}Clostridiales; | 0.0% | 0.4% | 0.7% | 0.4% |
| | f_{——}Veillonellaceae; g_{——} | | | | |
| | k_{——}Bacteria; p_{——}Firmicutes; c_{——}Clostridia; o_{——}Clostridiales; | 0.0% | 0.5% | 0.3% | 0.0% |
| | f_{——}[Mogibacteriaceae]; g_{——} | | | | |
| | k_{——}Bacteria; p_{——}Firmicutes; c_{——}Erysipelotrichi; o_{——}Erysipelotrichales; | 14.4% | 2.0% | 1.4% | 0.2% |
| | f_{——}Erysipelotrichaceae; g_{——} | | | | |
| | k_{——}Bacteria; p_{——}Firmicutes; c_{——}Erysipelotrichi; o_{——}Erysipelotrichales; | 0.5% | 7.7% | 5.3% | 14.3% |
| | f_{——}Erysipelotrichaceae; g_{——}Allobaculum; | | | | |
| | k_{——}Bacteria; p_{——}Proteobacteria; c_{——}Betaproteobacteria; | 1.9% | 0.0% | 0.0% | 1.1% |
| | o_{——}Burkholderiales; f_{——}Alcaligenaceae; g_{——}Sutterella | | | | |
| | k_{——}Bacteria; p_{——}Proteobacteria; c_{——}Deltaproteobacteria; | 0.7% | 7.0% | 18.4% | 1.9% |
| | o_{——}Desulfovibrionales; f_{——}Desulfovibrionaceae; g_{——} | | | | |
| | k_{——}Bacteria; p_{——}Proteobacteria; c_{——}Deltaproteobacteria; | 0.0% | 0.8% | 1.0% | 0.2% |
| | o_{——}Desulfovibrionales; f_{——}Desulfovibrionaceae; g_{——}Desulfovibrio | | | | |
| | k_{——}Bacteria; p_{——}Proteobacteria; c_{——}Epsilonproteobacteria; | 0.0% | 0.3% | 0.2% | 0.6% |
| | o_{——}Campylobacterales; f_{——}Helicobacteraceae; g_{——} | | | | |
| | k_{——}Bacteria; p_{——}Proteobacteria; c_{——}Epsilonproteobacteria; | 0.0% | 1.9% | 0.3% | 0.0% |
| | o_{——}Campylobacterales; f_{——}Helicobacteraceae; g_{——}Flexispira | | | | |
| | k_{——}Bacteria; p_{——}Proteobacteria; c_{——}Epsilonproteobacteria; | 0.0% | 0.4% | 0.2% | 0.1% |
| | o_{——}Campylobacterales; f_{——}Helicobacteraceae; g_{——}Helicobacter | | | | |
| | k_{——}Bacteria; p_{——}Proteobacteria; c_{——}Gammaproteobacteria; | 2.6% | 0.0% | 0.1% | 3.6% |
| | o_{——}Enterobacteriales; f_{——}Enterobacteriaceae; g_{——} | | | | |
| | k_{——}Bacteria; p_{——}Proteobacteria; c_{——}Gammaproteobacteria; | 13.2% | 0.1% | 0.0% | 0.0% |
| | o_{——}Enterobacteriales; f_{——}Enterobacteriaceae; g_{——}Klebsiella | | | | |
| | k_{——}Bacteria; p_{——}TM7; c_{——}TM7-3; o_{——}CW040; f_{——}F16; g_{——} | 0.0% | 0.9% | 0.7% | 0.8% |
| | k_{——}Bacteria; p_{——}Tenericutes; c_{——}Mollicutes; o_{——}RF39; f_{——}; g_{——} | 0.0% | 0.0% | 0.2% | 0.2% |
| | k_{——}Bacteria; p_{——}Verrucomicrobia; c_{——}Verrucomicrobiae; | 29.1% | 0.9% | 0.0% | 0.0% |
| | o_{——}Verrucomicrobiales; f_{——}Verrucomicrobiaceae; g_{——}Akkermansia | | | | |

As evident from the table above, both the high-dose administration group (D-H, 100 mg/kg) and the medium-dose administration group (D-L, 75 mg/kg) exhibit significant enhancements in the number of microflora within the genus Akkermansia. Notably, the high-dose group demonstrated a remarkable increase rate of 29.1% compared to the control group.

In subsequent experiments, a detailed analysis and testing were conducted on the strains included in the genus Akkermansia.

TABLE 7
Effects of Forsythia suspensa leaf extract on the species level of intestinal flora

| | | Total | | | |
|---|---|---|---|---|---|
| | | D-H | D-M | D-L | CW |
| Legend | Taxonomy | % | % | % | % |
| | k_{——}Bacteria; p_{——}Actinobacteria; c_{——}Coriobacteriia; | 0.0% | 0.4% | 0.6% | 0.7% |
| | o_{——}Coriobacteriales; f_{——}Coriobacteriaceae; g_{——}; s_{——} | | | | |
| | k_{——}Bacteria; p_{——}Actinobacteria; c_{——}Coriobacteriia; | 0.1% | 1.6% | 1.2% | 0.7% |
| | o_{——}Coriobacteriales; f_{——}Coriobacteriaceae; g_{——}Adlercreutzia; s_{——} | | | | |
| | k_{——}Bacteria; p_{——}Bacteroidetes; c_{——}Bacteroidia; o_{——}Bacteroidales; f_{——}; g_{——}; s_{——} | 0.1% | 0.2% | 1.3% | 0.4% |
| | k_{——}Bacteria; p_{——}Bacteroidetes; c_{——}Bacteroidia; o_{——}Bacteroidales; | 12.8% | 0.7% | 0.6% | 0.2% |
| | f_{——}Bacteroidaceae; g_{——}Bacteroides; s_{——} | | | | |
| | k_{——}Bacteria; p_{——}Bacteroidetes; c_{——}Bacteroidia; o_{——}Bacteroidales; | 0.0% | 0.3% | 0.2% | 0.1% |
| | f_{——}Bacteroidaceae; g_{——}Bacteroides; s_{——}acidifaciens | | | | |
| | k_{——}Bacteria; p_{——}Bacteroidetes; c_{——}Bacteroidia; o_{——}Bacteroidales; | 1.8% | 0.0% | 0.0% | 0.0% |
| | f_{——}Porphyromonadaceae; g_{——}Parabacteroides; s_{——} | | | | |
| | k_{——}Bacteria; p_{——}Bacteroidetes; c_{——}Bacteroidia; o_{——}Bacteroidales; | 0.0% | 0.5% | 0.6% | 0.2% |
| | f_{——}Rikenellaceae; g_{——}; s_{——} | | | | |
| | k_{——}Bacteria; p_{——}Bacteroidetes; c_{——}Bacteroidia; o_{——}Bacteroidales; | 0.1% | 3.5% | 5.2% | 21.7% |
| | f_{——}S24-7; g_{——}; s_{——} | | | | |
| | k_{——}Bacteria; p_{——}Bacteroidetes; c_{——}Bacteroidia; o_{——}Bacteroidales; | 0.0% | 0.0% | 1.0% | 0.4% |
| | f_{——}[Odoribacteraceae]; g_{——}Odoribacter; s_{——} | | | | |
| | k_{——}Bacteria; p_{——}Bacteroidetes; c_{——}Bacteroidia; o_{——}Bacteroidales; | 0.0% | 0.0% | 0.1% | 3.4% |
| | f_{——}[Paraprevotellaceae]; g_{——}[Prevotella]; s_{——} | | | | |
| | k_{——}Bacteria; p_{——}Deferribacteres; c_{——}Deferribacteres; o_{——}Deferribacterales; | 0.1% | 1.4% | 0.5% | 0.1% |
| | f_{——}Deferribacteraceae; g_{——}Mucispirillum; s_{——}schaedleri | | | | |
| | k_{——}Bacteria; p_{——}Firmicutes; c_{——}Bacilli; o_{——}Lactobacillales; | 0.1% | 2.2% | 6.2% | 5.8% |
| | f_{——}Lactobacillaceae; g_{——}Lactobacillus; s_{——} | | | | |
| | k_{——}Bacteria; p_{——}Firmicutes; c_{——}Bacilli; o_{——}Lactobacillales; | 0.0% | 0.1% | 5.6% | 0.4% |
| | f_{——}Lactobacillaceae; g_{——}Lactobacillus; s_{——}reuteri | | | | |
| | k_{——}Bacteria; p_{——}Firmicutes; c_{——}Bacilli; o_{——}Lactobacillales; | 0.1% | 7.8% | 3.4% | 0.0% |
| | f_{——}Streptococcaceae; g_{——}Lactococcus; s_{——} | | | | |
| | k_{——}Bacteria; p_{——}Firmicutes; c_{——}Clostridia; o_{——}Clostridiales; f_{——}; g_{——}; s_{——} | 0.1% | 19.6% | 20.4% | 22.9% |
| | k_{——}Bacteria; p_{——}Firmicutes; c_{——}Clostridia; o_{——}Clostridiales; | 2.6% | 3.1% | 1.9% | 5.0% |
| | f_{——}Lachnospiraceae; g_{——}; s_{——} | | | | |
| | k_{——}Bacteria; p_{——}Firmicutes; c_{——}Clostridia; o_{——}Clostridiales; | 0.8% | 0.0% | 0.0% | 0.0% |
| | f_{——}Lachnospiraceae; g_{——}Blautia; s_{——} | | | | |
| | k_{——}Bacteria; p_{——}Firmicutes; c_{——}Clostridia; o_{——}Clostridiales; | 2.5% | 0.1% | 0.0% | 0.0% |
| | f_{——}Lachnospiraceae; g_{——}Blautia; s_{——}producta | | | | |
| | k_{——}Bacteria; p_{——}Firmicutes; c_{——}Clostridia; o_{——}Clostridiales; | 0.0% | 1.0% | 0.2% | 0.2% |
| | f_{——}Lachnospiraceae; g_{——}Coprococcus; s_{——} | | | | |
| | k_{——}Bacteria; p_{——}Firmicutes; c_{——}Clostridia; o_{——}Clostridiales; | 0.1% | 5.9% | 1.2% | 1.1% |
| | f_{——}Lachnospiraceae; g_{——}Dorea; s_{——} | | | | |
| | k_{——}Bacteria; p_{——}Firmicutes; c_{——}Clostridia; o_{——}Clostridiales; | 0.1% | 0.2% | 4.5% | 0.0% |
| | f_{——}Lachnospiraceae; g_{——}Dorea; s_{——}formicigenerans | | | | |
| | k_{——}Bacteria; p_{——}Firmicutes; c_{——}Clostridia; o_{——}Clostridiales; | 0.9% | 0.5% | 0.9% | 0.1% |
| | f_{——}Lachnospiraceae; g_{——}[Ruminococcus]; s_{——} | | | | |
| | k_{——}Bacteria; p_{——}Firmicutes; c_{——}Clostridia; o_{——}Clostridiales; | 0.0% | 4.3% | 1.4% | 2.6% |
| | f_{——}Lachnospiraceae; g_{——}[Ruminococcus]; s_{——}gnavus | | | | |
| | k_{——}Bacteria; p_{——}Firmicutes; c_{——}Clostridia; o_{——}Clostridiales; | 11.9% | 8.3% | 1.9% | 2.3% |
| | f_{——}Ruminococcaceae; g_{——}; s_{——} | | | | |
| | k_{——}Bacteria; p_{——}Firmicutes; c_{——}Clostridia; o_{——}Clostridiales; | 0.7% | 9.1% | 8.5% | 2.8% |
| | f_{——}Ruminococcaceae; g_{——}Oscillospira; s_{——} | | | | |
| | k_{——}Bacteria; p_{——}Firmicutes; c_{——}Clostridia; o_{——}Clostridiales; | 0.1% | 1.2% | 0.5% | 1.6% |
| | f_{——}Ruminococcaceae; g_{——}Ruminococcus; s_{——} | | | | |
| | k_{——}Bacteria; p_{——}Firmicutes; c_{——}Clostridia; o_{——}Clostridiales; | 0.0% | 0.4% | 0.7% | 0.4% |
| | f_{——}Veillonellaceae; g_{——}; s_{——} | | | | |
| | k_{——}Bacteria; p_{——}Firmicutes; c_{——}Erysipelotrichi; o_{——}Erysipelotrichales; | 14.4% | 2.0% | 1.4% | 0.2% |
| | f_{——}Erysipelotrichaceae; g_{——}; s_{——} | | | | |
| | k_{——}Bacteria; p_{——}Firmicutes; c_{——}Erysipelotrichi; o_{——}Erysipelotrichales; | 0.5% | 7.7% | 5.3% | 14.3% |
| | f_{——}Erysipelotrichaceae; g_{——}Allobaculum; s_{——} | | | | |
| | k_{——}Bacteria; p_{——}Proteobacteria; c_{——}Betaproteobacteria; | 1.9% | 0.0% | 0.0% | 1.1% |
| | o_{——}Burkholderiales; f_{——}Alcaligenaceae; g_{——}Sutterella; s_{——} | | | | |
| | k_{——}Bacteria; p_{——}Proteobacteria; c_{——}Deltaproteobacteria; | 0.7% | 7.0% | 18.4% | 1.9% |
| | o_{——}Desulfovibrionales; f_{——}Desulfovibrionaceae; g_{——}; s_{——} | | | | |
| | k_{——}Bacteria; p_{——}Proteobacteria; c_{——}Deltaproteobacteria; | 0.0% | 0.8% | 1.0% | 0.2% |
| | o_{——}Desulfovibrionales; f_{——}Desulfovibrionaceae; g_{——}Desulfovibrio; s_{——} | | | | |
| | k_{——}Bacteria; p_{——}Proteobacteria; c_{——}Epsilonproteobacteria; | 0.0% | 0.3% | 0.2% | 0.6% |
| | o_{——}Campylobacterales; f_{——}Helicobacteraceae; g_{——}; s_{——} | | | | |
| | k_{——}Bacteria; p_{——}Proteobacteria; c_{——}Epsilonproteobacteria; | 0.0% | 1.9% | 0.3% | 0.0% |
| | o_{——}Campylobacterales; f_{——}Helicobacteraceae; g_{——}Flexispira; s_{——} | | | | |
| | k_{——}Bacteria; p_{——}Proteobacteria; c_{——}Gammaproteobacteria; | 2.6% | 0.0% | 0.1% | 3.6% |
| | o_{——}Enterobacteriales; f_{——}Enterobacteriaceae; g_{——}; s_{——} | | | | |
| | k_{——}Bacteria; p_{——}Proteobacteria; c_{——}Gammaproteobacteria; | 13.2% | 0.1% | 0.0% | 0.0% |
| | o_{——}Enterobacteriales; f_{——}Enterobacteriaceae; g_{——}Klebsiella; s_{——} | | | | |
| | k_{——}Bacteria; p_{——}TM7; c_{——}TM7-3; o_{——}CW040; f_{——}F16; g_{——}; s_{——} | 0.0% | 0.9% | 0.7% | 0.8% |
| | k_{——}Bacteria; p_{——}Verrucomicrobia; c_{——}Verrucomicrobiae; | 29.1% | 0.9% | 0.0% | 0.0% |
| | o_{——}Verrucomicrobiales; f_{——}Verrucomicrobiaceae; g_{——}Akkermansia; | | | | |
| | s_{——}muciniphila | | | | |

As evident from the table above, both the high-dose administration group (D-H, 100 mg/kg) and the medium-dose administration group (D-L, 75 mg/kg) exhibit significant enhancements in the number of microflora within the species Akkermansia muciniphila. Notably, the high-dose group demonstrated a remarkable increase rate of 29.1% compared to the control group.

The experimental results above indicated a relatively low relative content of AKK bacteria in the intestinal tract of mice. Through analyses at various taxonomic levels, including phylum, class, order, family, genus, and species, it was observed that the AKK bacteria content in the intestinal tract of the normal group without drug administration was 0. After high, medium, and low-dose administrations, the AKK bacteria content in the intestinal tract remained at 0 for the low-dose group (50 mg/kg), while for the high-dose (100 mg/kg) and medium-dose (75 mg/kg) groups, the AKK bacteria content was 29.1% and 0.9%, respectively. This confirms that Forsythia suspensa leaf extract effectively enhances the abundance of AKK bacteria in the intestinal tract, with the abundance increasing with dosage. The effect was particularly significant at a dosage of 100 mg/kg.

The aforementioned embodiments represent preferences of the present invention and are not intended to restrict it. Any modifications, equivalent replacements, improvements, etc., made within the spirit and principles of the present invention, are included in the scope of protection of the invention.

What is claimed is:

1. A preparation method for a Forsythia suspensa leaf extract, comprising:
S1: taking Forsythia suspensa leaves, grinding them, adding a solvent at a temperature above 90° C. for 10 to 40 min, filtering and then adding an extractant for extraction for more than 6 hours to obtain a first product;
S2: filtering the first product, taking a supernatant, adsorbing, eluting, and concentrating an eluate under reduced pressure to obtain a second product;
S3: dissolving the second product in a water, carrying out chromatography, then eluting, collecting an eluate, concentrating under reduced pressure to obtain the Forsythia suspensa leaf extract.

2. The preparation method of claim 1, wherein in step S1, the solvent is water and the extractant is methanol, ethanol or propanol.

3. The preparation method of claim 1, wherein in step S2, the process of the eluting is as follows: first eluting with water, then eluting with 30-60% ethanol, combining with an ethanol elution.

4. The preparation method of claim 1, wherein the volume ratio of the second product to the water in step S3 is 1-10:5-50.

5. A Forsythia suspensa leaf extract, wherein it is prepared by the preparation method of claim 4.

6. A use of the Forsythia suspensa leaf extract of claim 5 for preparing a drug that promotes the abundance of the bacteria species Akkermansia muciniphila or the bacteria genus Akkermansia in the intestinal tract of animals.

7. The use of claim 6, wherein the drug is an oral drug.

8. The use of claim 6, wherein the animals are mammal.

9. The use of claim 6, wherein dosage form of the drug is a paste, syrup, tablet or capsule.

10. The use of claim 7, wherein dosage form of the drug is a paste, syrup, tablet or capsule.

11. The use of claim 8, wherein dosage form of the drug is a paste, syrup, tablet or capsule.
